(19) United States
(12) Patent Application Publication
(10) Pub. No.: US 2023/0265373 A1
(43) Pub. Date: Aug. 24, 2023

(54) A Multi-Sample System for Engineered Tissue Strip Assays

(71) Applicant: Novoheart International Limited, Kowloon (HK)

(72) Inventors: Yosuke Kurokawa, Kwun Tong (HK); Eugene K. Lee, Kwun Tong (HK); David D. Tran, Kwun Tong (HK); Ronald A. Li, Kwun Tong (HK)

(21) Appl. No.: 18/016,254

(22) PCT Filed: Jul. 14, 2021
(86) PCT No.: PCT/US2021/041656

§ 371 Date: Jan. 13, 2023

Related U.S. Application Data
(60) Provisional application No. 63/051,447, filed on Jul. 14, 2020.

(51) Int. Cl. C12M 3/00; C12M 1/42; C12M 1/34

(52) U.S. Cl. CPC C12M 21/08; C12M 35/04; C12M 41/00

(57) ABSTRACT

A multi-sample system for engineered tissue strip assay includes a tissue module enabled with a set of channels each configured to house one or more tissue strips, each tissue strip is in contact with a sensor at one end and a tissue lengthening mechanism at the other end, the sensor is displaced or deformed when the tissue strip exerts force against the sensor during the lengthening or contraction of each tissue strip; and a detection system configured to capture change in the sensor in contact with the tissue strips during lengthening or contraction of at least one of the tissue strips.

### CROSS REFERENCE TO RELATED APPLICATIONS

This application claims the benefit of U.S. provisional patent application 63/051,447 filed Jul. 14, 2021, the entire contents of which is incorporated by reference herein.

### TECHNICAL FIELD

The present application relates to multi-sample platforms, and particularly relates to assays using engineered tissue strips.

### BACKGROUND

Engineered tissue strips have been used in a variety of assays in the field of biotechnology, for example in drug screening, biomechanical characterization, tissue conditioning and the like. In these assays, the engineered tissue strips are often displaced (i.e., stretched or contracted) for the purposes of making different observations.

Taking drug screening as an example, several drug screening platforms have been developed using human pluripotent stem cell (hPSC)-derived cardiomyocytes (CMs). One such platform is the cardiac tissue strip (CTS) assay, which is a three-dimensional cardiac construct that is uniaxially loaded by anchoring the two ends of the CTS. Through cell-mediated compaction, an aligned, elongated tissue is formed, which produces both passive and active tension. CTS assays have been used to validate physiological responses to a wide range of drugs, including some that have failed traditional preclinical screening methods.

Applying physiological loads to displace (i.e., stretch or contract) the tissue is advantageous for observing specific disease phenotypes and drug responses. However, typical systems for displacing muscle tissues are low-throughput (single-tissue) assays that are cost-prohibitive for scaling.

There are two major technical challenges in creating an assay platform that can displace the engineered tissue strips and measure forces from multiple tissues simultaneously. Firstly, the platform must enable the formation, culture, displacing, and electrical pacing of multiple tissues. The platform has to parallelize the tasks across multiple tissues for increased efficiency and throughput. The design should also allow for long-term culture of the tissues, which requires a sterile environment with temperature and CO₂ control. Secondly, the relatively weak forces generated by the tissues (active and/or passive) must be measured in a cost-effective manner. Few commercial systems offer force transducers with sufficiently high sensitivity and accuracy for tissue engineering applications, and commercial systems that rely on such specialized force transducers are expensive and cost-prohibitive for scaling.

Thus, there is a need for a multi-sample system for engineered tissue strip assays which are sensitive, accurate, cost-effective, and scalable.

### SUMMARY

This summary is provided to introduce concepts related to a multi-sample system for scalable engineered tissue strip assays. This summary is not intended to identify essential features of the claimed subject matter nor is it intended for use in determining or limiting the scope of the claimed subject matter.

The present application provides a multi-sample system for engineered tissue strip assay including: an engineered tissue strip module enabled with a set of channels, wherein each channel is configured to house an engineered tissue strip from a set of engineered tissue strips, wherein each engineered tissue strip is in contact with a sensor at one end and a tissue lengthening mechanism at the other end, wherein the tissue lengthening mechanism is configured to lengthen or contract at least one of the engineered tissue strips, wherein the sensor is displaced or deformed when the engineered tissue strip exerts force against the sensor during the lengthening or contraction of each of the engineered tissue strips; and a detection system configured to capture change in the sensor in contact with the engineered tissue strip, during lengthening or contraction of at least one of the engineered tissue strips.

In some embodiments, the detection system includes a mirror array system including an imaging platform with a camera and a set of mirrors, wherein the camera is configured to capture a video of at least one of the engineered tissue strips and the sensor in contact with the engineered tissue strip, through the set of mirrors, during the lengthening or contraction of at least one of the engineered tissue strips.

In some embodiments, the video is processed using an image-processing based kymograph for determining characteristic properties of each of the engineered tissue strips from the set of engineered tissue strips or the subset of engineered tissue strips.

In some embodiments, the multi-sample system further includes a bioreactor for housing the engineered tissue strip module and the mirror array system, wherein the bioreactor provides environmental control and monitoring of one or more environmental conditions of interest including temperature, carbon dioxide (CO₂), relative humidity, and oxygen (O₂) concentration in the bioreactor.

In some embodiments, the tissue lengthening mechanism is configured to control linear displacement.

In some embodiments, the tissue lengthening mechanism includes a linear actuator, a micrometer, a motor, a ratchet, a pinion, a camshaft, or a mechanical linkage integrated into the bioreactor.

In some embodiments, the engineered tissue strip module enables formation, culturing, and controlling of at least one of the engineered tissue strips, and wherein each channel is enabled with an inlet port and an outlet port, wherein a perfusion system is configured for fluid circulation (such as cell culture media) in each channel through the inlet port and the outlet port, and wherein each channel is recessed to allow for the fluid to flow around the engineered tissue strips to aid in initial compaction of the tissue gel mixture, wherein volume of the fluid is limited and contained in specific part of the set of channels with the sensor, wherein at least one of the set of channels are connected upstream and downstream to common areas for inlet and outlet of tissue culture media, and wherein the common areas are connected to the perfusion system through a common inlet port and common outlet port.

In some embodiments, the set of channels is isolated from each other with a dedicated inlet and outlet for each channel, and wherein the dedicated inlet and outlet of each of the channel connects to a same or different perfusion system.

In some embodiments, one end of each of the engineered tissue strips is anchored to a rigid body that the engineered tissue strips cannot displace or deform, wherein the rigid body is selected from a shape including needle, rod, pin, post, or anchor, and wherein the rigid body is connected to a movable block of a rail system to enable lengthening of the engineered tissue strips.

In some embodiments, the rigid body is enabled with an electrode component configured to electrically pace the set of engineered tissue strips or the subset of engineered tissue strips, wherein the electrode component is composed of a conductive material or an array of electrodes positioned on each side of the engineered tissue strips to apply voltage for electrically pacing the engineered tissue strips, and wherein the rigid body is enabled to aid in the anchoring of the engineered tissue strips.

In some embodiments, the sensor is a geometrically defined material that displaces and/or deforms when the engineered tissue strip exerts force on the sensor, and wherein the displacement and/or deformation of the sensor is monitored by the detection system to measure the lengthening or contraction of the engineered tissue strip, wherein the sensor is a passive sensor or an active sensor.

In some embodiments, the engineered tissue strip module is made of a plurality of sheets or layers, wherein a sheet or layer forming a bottom of the engineered tissue strip module is optically transparent to allow potential optical imaging and/or optical mapping by the mirror array system, and wherein any leakage of fluid in between the plurality of sheets or layers is prevented by a sealing member incorporated in between the plurality of sheets or layers.

In some embodiments, the multi-sample system includes an alignment component to align each of the sensors in the channels of the engineered tissue strip module, wherein the alignment component includes a top frame and a bottom frame configured to accommodate the sensors in between the top frame and bottom frame, wherein the top frame, bottom frame and sensors have corresponding holes or registration mechanisms or markers that are used for alignment of the sensor in between the top frame and the bottom frame within the same plane, wherein a dowel pin is press-fitted into each of the holes to align the sensors, wherein the top frame of the alignment component have additional holes between every two sensors, wherein the additional holes act as inlet for tubes of the perfusion system, and wherein the additional holes directly feed into the channels of the engineered tissue strip module.

In some embodiments, the sensors are directly embedded into the wall of the engineered tissue strip module when at least one region of the sensors is to be isolated from the fluid reservoir.

In some embodiments, the tissue lengthening mechanism is configured to displace the movable block forward and backward to allow lengthening of the engineered tissue strip, wherein the rail system includes one or more rods restricting movement of the movable block to one axis.

In some embodiments, the tissue lengthening mechanism is a linear actuator, wherein the linear actuator is connected to a computer and controlled using programmed instructions stored within the computer, wherein position information of a linear actuator rod, of the linear actuator, is measured by executing the programmed instructions for a predefined time period, wherein the position information is used to calculate tissue length and percentage of tissue stretch.

In some embodiments, at least one camera of the imaging platform is configured to optically measure twitch forces of the set of engineered tissue strips simultaneously, wherein at least one camera is enabled with microscopic lenses oriented horizontally towards the set of mirrors that direct toward the engineered tissue strips, wherein the set of mirrors are arranged to enable the camera to capture multiple engineered tissue strips simultaneously, wherein the mirror arrangement enables capturing separate views of the engineered tissue strips located apart and combine the views together into a single image captured by the camera, wherein the set of mirrors are positioned to have equal focal distance for all of the engineered tissue strips to ensure all engineered tissue strips are in the same focal plane, wherein the set of mirrors are mounted over sliding elements that adjust the distance between the camera and the engineered tissue strip, and wherein the sliding elements enable a focusing mechanism to ensure that the engineered tissue strips are in focus with the camera.

In some embodiments, a multi-channel peristaltic pump is used to drive fluid flow into and suction out of the engineered tissue strip module, wherein media is pumped into the engineered tissue strip module at flow rates ranging from 0.001 to 10 mL/min per channel, wherein liquid height in each channel is set by adjusting the position of an outlet tubing, and wherein the outlet flow is recycled back to an inlet of the fluid reservoir, redirected for collection or disposed of

In some embodiments, the imaging platform is enabled with an inverted optical mapping system, wherein the inverted optical mapping system includes an excitation source, filter cubes, mirrors, camera, and lens, wherein the inverted optical mapping system is configured to measure tissue properties including conduction velocity of the engineered tissue strips, wherein the engineered tissue strips exhibit fluorescence or bioluminescence.

In some embodiments, the engineered tissue strips exhibit fluorescence or bioluminescence through the use of fluorescent or bioluminescent dyes or the expression of transgenes encoding fluorescent or bioluminescent proteins.

In some embodiments, each of the sensors is marked with a marker, wherein the marker is detectable when excited by a fluorescent light source, wherein the marker is trackable during conduction velocity measurements, and wherein the marker is tracked simultaneously to capture sensor movement, wherein the sensor movement is used for computing applied force by the tissue.

In some embodiments, the multi-sample system further includes program executable instructions for controlling the linear actuator, heating unit, thermocouple, CO₂ sensor solenoid valve, and cameras, the program executable instructions including instructions to: create markers on the images acquired from the camera to allow a user to set the linear actuator to the engineered tissue strips at an unstretched length, calculate tissue length, and percentage stretch based on the linear actuator position, and save and convert the images acquired from the camera into a video file using a semi-automated file naming scheme.

In some embodiments, the image-processing based kymograph includes the steps of: defining a region-of-interest (ROI) corresponding to each of the engineered tissue strips from the set of engineered tissue strips or the subset of engineered tissue strips, in a frame of the video, wherein the ROI is selected along reference marks, wherein the reference marks corresponds to stationary objects in the bioreactor; applying binary thresholding on each pixel in each of the ROIs to convert the ROI into a binary pixel representation; defining a primary axis of the engineered tissue strip contraction and a target region along the primary axis of the engineered tissue strip contraction in the binary pixel representation of each of the ROIs; applying Gaussian filter and interpolation on to the target region, in a spatial manner, of each video frame of the video, to achieve a sub-pixel resolution corresponding to the target region in each video frame; generating a signal over time based on the sub-pixel resolution captured from each of the video frames, wherein the signal represents the engineered tissue strip position over time; and generating a representative signal by averaging all the signals generated from each target region in the video frame.

### BRIEF DESCRIPTION OF THE DRAWINGS

The detailed description is described with reference to the accompanying figures. In the figures, the left-most digit(s) of a reference number identifies the figure in which the reference number first appears. The same numbers are used throughout the drawings to refer to like features and components.

FIG. 1A-B illustrates a 3D model of a multi-sampling system (FIG. 1A) and a mirror array system (FIG. 1B) in the multi-sampling system, in accordance with an embodiment of the present application.

FIG. 2A-D illustrates a 3D model of an engineered tissue strip module (FIG. 2A) and placement of an engineered tissue strip in each channel of the engineered tissue strip module (FIG. 2B-D), in accordance with an embodiment of the present application.

FIG. 3A-B illustrates an alignment component (FIG. 3A) for insertion of a set of sensors in the engineered tissue strip module (FIG. 3B), in accordance with an embodiment of the present application.

FIG. 4A-B illustrates a sensor embedded in the engineered tissue strip module (FIG. 4A) and gaskets for sealing the sensor between two layers of the engineered tissue strip module (FIG. 4B), in accordance with an embodiment of the present application.

FIG. 5 illustrates a 3D model of a tissue lengthening mechanism, in accordance with an embodiment of the present application.

FIG. 6A-C illustrates a 3D model of the mirror array system shown from different perspectives, in accordance with an embodiment of the present application.

FIG. 7 illustrates an electrode component, in accordance with an embodiment of the present application.

FIG. 8 illustrated a top view of a perfusion system, in accordance with an embodiment of the present application.

FIG. 9A-B illustrates a 3D model of an optical mapping system from different perspectives, in accordance with an embodiment of the present application.

FIG. 10A-B illustrates a user interface of a custom software for the bioreactor (FIG. 10A) and a kymograph-based image processing technique enabled by the custom software (FIG. 10B), in accordance with an embodiment of the present application.

FIG. 11 illustrates sensors used within the bioreactor module. PDMS inserts were used during the initial tissue formation (left). The tissue anchored around the opening of the sensor end (right).

FIG. 12 illustrates graphs representing a positive length-tension relationship of a cardiac tissue strip, in accordance with an embodiment of the present application.

FIG. 13 illustrates a variable length-tension relationship produced using cardiomyocytes from three separate human stem cell lines.

FIG. 14 illustrates a graph representing a concentration-dependent negative inotropic response to nifedipine.

### DETAILED DESCRIPTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments.

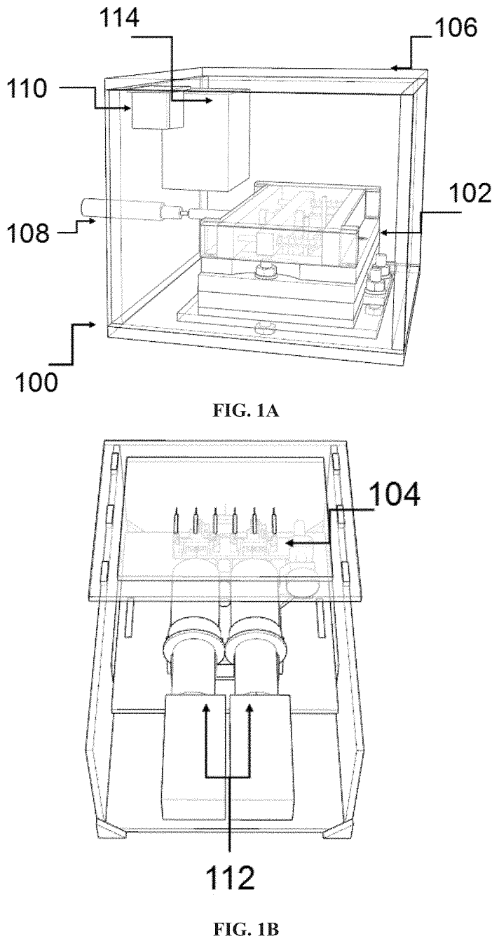

Referring now to FIG. 1A and 1B, a 3D model of a multi-sampling system 100 to enable simultaneous displacement of multiple engineered tissue strips is disclosed, in accordance with embodiments of the present application. The engineered tissue strips may be any strip of soft engineered tissue such as cardiac (atrial, ventricular, purkinje, valve, pericardium), artery, vein, lymph, skeletal muscle, tendon, ligament, smooth muscle, and the like. It should be understood that the multi-sample system 100 is not limited to the use in drug screening, but also for disease modelling, screening of other types of non-drug therapeutics (cells, genes, biomaterials, etc), and may also be used for tissue conditioning (chronic stretch protocols) without screening.

The multi-sampling system 100 may include an engineered tissue strip module 102, a detection system, a bioreactor 106, a tissue lengthening mechanism 108, and a perfusion system (not shown). In FIG. 1A and 1B, the detection system is illustrated as a mirror array system 104. However, one skilled in the art would appreciate that other means can be used as the detection system so long as the detection system is capable of capturing change in the sensor in contact with the engineered tissue strip, during lengthening or contraction of at least one of the engineered tissue strips. The bioreactor 106 is the enclosure that houses the engineered tissue strip module 102 and provides the necessary environmental monitoring and feedback control in terms of environmental conditions of interest such as temperature, CO₂, relative humidity, and oxygen (O₂) concentration. The bioreactor 106 may include a heating unit 114 and a CO₂ module 110. The heating unit 114 may include a heating element, an electrical heat distributing element, and a temperature measuring element. In some embodiments, the heating element is an electrical heating element. In some embodiments, the electrical heating element may have a power between 1 to 10,000 Watt. In some embodiments, the electrical heating element may have a power between 100 to 250 Watt. In some embodiments, the electrical heat distributing element may be an electrical fan. In some embodiments, the temperature measuring element is a thermocouple. It should be understood that the heating unit 114 is not limited to three specific parts, and other combinations and alternatives of this setup can be utilized so long as heat is generated and dissipated uniformly in the bioreactor 106. The heating unit 114 may be configured to maintain the internal temperature of the bioreactor 106 at any desirable temperature. In some embodiments, the internal temperature of the bioreactor may be 37° C. with a standard deviation of half a degree. This temperature range may be altered programmatically.

The CO₂ module 110 may be composed of a CO₂ sensor (not shown), and a valve (not shown) that controls the inflow of CO₂ from a gas source into the bioreactor 106 to achieve a desired gas mixture of any concentration, such as 0.1% CO₂, 0.5% CO₂, 1.5% CO₂, 2% CO₂, 2.5% CO₂, 3% CO₂, 3.5% CO₂, 4% CO₂, 4.5% CO₂, 5% CO₂, 5.5% CO₂, 6% CO₂, 6.5% CO₂, 7% CO₂, 7.5% CO₂, 8% CO₂, 8.5% CO₂, 9% CO₂, 9.5% CO₂, 10% CO₂, 15% CO₂, or 20% CO₂. The valve may be, but is not limited to, a solenoid valve.

Both the heating unit 114 and the CO₂ module 110 may be programmatically controlled using a feedback process control loop. Further, the mirror array system 104 may include a set of mirrors and at least one camera. It should be understood that each mirror from the set of mirrors may be configured to reflect light from each channel in the engineered tissue strip module 102 towards the camera such that the camera can simultaneously capture images/video of each channel in the engineered tissue strip module 102. It should be understood that more than one camera with a separate array/set of mirrors may be used to capture images/videos of distinct set of engineered tissue strips placed in the same engineered tissue strip module. In an example, one camera and a set of mirrors may be configured to optically capture the contractions of three different engineered tissue strips simultaneously. If a pair of cameras 112, as depicted in FIG. 1B, is used, simultaneous acquisition of 6 engineered tissue strips can be achieved. The number of cameras and mirrors may be selected as per requirement of the engineered tissue strip assay.

In some embodiments, the tissue lengthening mechanism 108 is provided in order to stretch the engineered tissue strips in the engineered tissue strip module 102. In some embodiments, the tissue lengthening mechanism 108 may be selected from a linear actuator, or any component that can control linear displacement such as a micrometer, a motor, a ratchet, a pinion, a camshaft, or a mechanical linkage. In one embodiment, the linear actuator is a Model X-NA08A25-E09 by Zaber. The tissue lengthening mechanism 108 may be mounted directly onto the wall of the bioreactor 106. The engineered tissue strip module 102 may also contain an electrode component (not shown) to electrically pace the tissue strips in the engineered tissue strip module 102 at a desired frequency, amplitude, duration, or whatever shape of electrical stimulus is desired. Further, the engineered tissue strip module 102 may have separate inlet and outlet ports for each channel in the engineered tissue strip module 102 for the integration of a perfusion system (not shown). Alternately, the engineered tissue strip module may have a common inlet and a common outlet port for the integration of a perfusion system (not shown). Specific details of the fabrication, setup and utility of the module are described further below.

Furthermore, the mirror array system 104 may be used to simultaneously measure the characteristics (contractile and mechanical properties) of the engineered tissue strips by tracking the displacement of a sensor corresponding to each engineered tissue strip in the engineered tissue strip module 102. The sensor may be a geometrically defined (e.g. having the geometry including but not limited to sigmoidal, zig-zag, flexure, thin rectangular, or coiled) and well characterized material (e.g. the material including but not limited to PDMS, silicone, polyurethane, ecoflex, or other biocompatible elastomer) that displaces or deforms when the engineered tissue strip is exerting force against the sensor placed in the engineered tissue strip module 102. Specific details of the fabrication, setup, and utility of the engineered tissue strip module 102 is described further below.

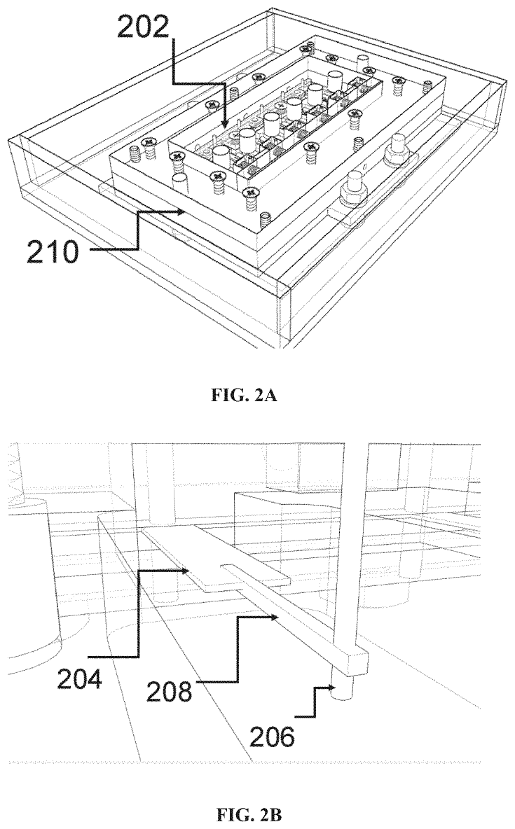

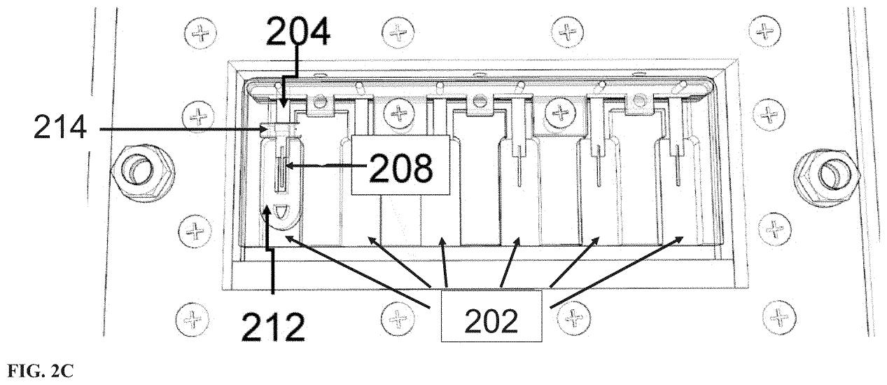

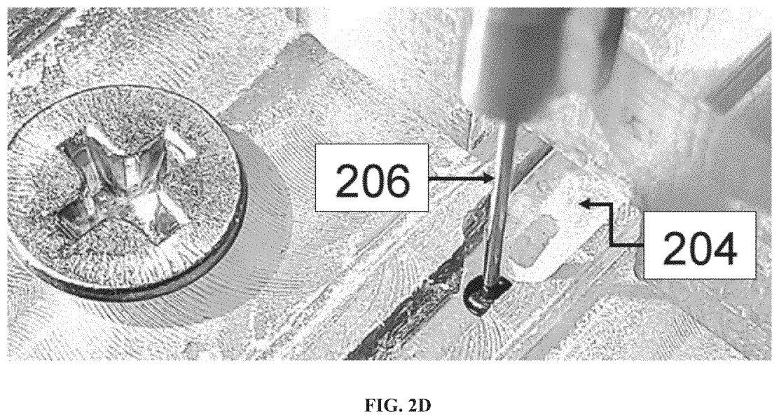

Referring now to FIG. 2A-D, a 3D model of the engineered tissue strip module 102 and placement of the engineered tissue strip 208 in each channel of the engineered tissue strip module 102 is illustrated, in accordance with embodiments of the present application. The engineered tissue strip module 102 is an apparatus wherein the engineered tissue strips are formed, cultured, and in some embodiments, displaced (e.g. lengthened or contracted). In some embodiments, the engineered tissue strip 208 may be a CTS, e.g. a human ventricular CTS (hvCTS). The engineered tissue strip module 102 may include a set of channels 202. Each channel from the set of channels 202 may be configured to house a sensor 204 and an engineered tissue strip 208 attached to the sensor 204. The channel allows for fluid or solution to flow through the channel. To aid the initial compaction of the tissue gel mixture, volume of the fluid may be limited and contained in a specific part of the channel with the help of a well insert 212 and sensor insert 214.

Furthermore, as illustrated in FIG. 2A, the engineered tissue strip module 102 may contain multiple parallel channels 202 for accommodating multiple different engineered tissue strips. In some embodiments, the number of channels may be two, three, four, five, six, seven, eight, nine, ten, or any integer greater than ten. The orientation of the channels may be altered depending on the requirements. For illustrative purpose, the engineered tissue strip module 102 in FIG. 2A and 2C contains six parallel channels for accommodating six discrete engineered tissue strips. The set of channels 202 may be connected upstream and downstream to common areas for inlet and outlet of tissue culture media. In other embodiments, the set of channels 202 may be completely isolated from each other with a dedicated inlet and outlet for each strip. Also, it is possible to rearrange the channels in different orientations other than parallel arrangement, for example as disclosed in WO2019106438A1 which is hereby incorporated by reference.

In some embodiments, one end of the engineered tissue strip 208 is anchored to a rigid body 206 such that the tissue is secured. In some embodiments, the rigid body may be a needle, rod, pin, post or anchor as shown in FIG. 2B. The rigid body may be made of stainless-steel, other metal, plastic, or other synthetic materials. The rigid body 206 for each engineered tissue strip may be in contact with a movable block of rail system (not shown) that allows for displacement of the engineered tissue strip 208. The rigid body 206 may further enable mounting of a conductive material or specifically have a cathode and anode part in which a voltage can be applied in a local region (e.g. point stimulation) to electrically pace the engineered tissue strip 208. The rigid body 206 may be of different geometrical designs, such as a hook, or contain attachments to aid in the anchoring of the engineered tissue strip 208. In one embodiment, the rigid body 206 may have a soft nitrile rubber O-ring. In some embodiments, in order to avoid physical damage to the engineered tissue strip by being punctured by the rigid body 206, the engineered tissue strip may be grown on the rigid body 206, such that the engineered tissue strip is integrated with the rigid body 206.

In some embodiments, the other end of each engineered tissue strip 208 may be anchored to a sensor 204. As previously described, the sensor 204 is a geometrically defined and well characterized material that displaces or deforms when the engineered tissue strip 208 is exerting force. The sensor 204 may be used to measure the lengthening or contraction of the engineered tissue strip 208 in either an active or passive manner.

In active mode, the sensing entails a signal being emitted by the sensor 204 via an external power source and a receiver that captures any changes to the sensor 204 as force is being applied onto the sensor by the engineered tissue strip 208. The sensor may be a piezo-resistive element in a soft material sensor, in which the voltage and resistance change based on the stretching or deformation of the sensor from the force exerted by the engineered tissue strip 208. On the other hand, when a passive mode is used, a passive sensor having a marker or characteristic that can be tracked by an external component may be used. The displacement of the marker due to engineered tissue strip 208 contraction can be converted into force by prior knowledge of the tensile properties of the sensor and understanding the behavior or response of the geometrical design under load. For example, in some embodiments, the sensor 204 may be composed of a defined silicone material fabricated in a rectangular prism or planar sheet shape. Hooke's law may be applied to the measured displacement of the marker before and after load to calculate force. In this embodiment, the sensor 204 is in-plane with the tissue mimicking a tendon-muscle relationship. In some embodiments, the sensor 204 may be a beam or rod that is perpendicular to the engineered tissue strip 208 and the force applied by the engineered tissue strip 208 is normal to the sensor 204. In this embodiment, the force can be estimated by the Euler-Bernoulli beam theory. For all embodiments, the stress-strain or equivalent force-displacement relationship of the sensor 204 may be altered by changing the tensile properties (e.g. Young's Modulus) or geometrical designs such as cross-sectional area of the sensor 204.

In some embodiments, the sensor 204 may comprise a sensor head and an attachment region engageable with the engineered tissue strip 208. The sensor head and the attachment region may be separated by a stretchable region. The stretchable region may comprise a plurality of stretchable portions spaced apart. The stretchable region may significantly increase the displacement of sensor head for a small amount of force generated by the stretching or contraction of the engineered tissue strip 208. In general, the sensor 204 may increase measurement resolution of forces generated by an engineered tissue strip 208 placed in the channel of the engineered tissue strip module 102. As a result, the movement of the sensor head can be easily captured by the imaging platform.

In some embodiments, the engineered tissue strip module 102 may be formed of a plurality of sheets or layers 210. In some embodiments, the sheets or layers 210 are transparent. In some embodiments, the sheets or layers are made of thermoplastic. In some embodiments, the sheets or layers are polycarbonate sheets or layers. The engineered tissue strip module may be fabricated by any conventional means, for example by stacking a set of polycarbonate sheets or layers together. Each polycarbonate sheet may be milled with a CNC router. The sheets or layers forming the bottom of the engineered tissue strip module 102 may be optically transparent to allow for potential optical imaging and/or optical mapping of the engineered tissue strip 208. To assemble the sheets or layers 210 together, any conventional mechanisms may be utilized. In some embodiments, the sheets or layers 210 may be assembled with screw holes that allow the sheets or layers 210 to be tightened together. To prevent any leakage of liquid in between the sheets or layers, a sealing member that can be reversibly removed and reapplied may be incorporated in between two neighboring sheets or layers. In some embodiments, the sealing member may be a gasket or a sealant. In some embodiments, the sealant is a stopcock grease. In some embodiments, the set of sheets or layers 210 may be fused together by adhesive (e.g. cyano-acrylate) or through a process of melting the sheets or layers 210 together with a chemical solution or high heat. In some embodiments, the engineered tissue strip module 102 may be fabricated as one solid piece using injection molding.

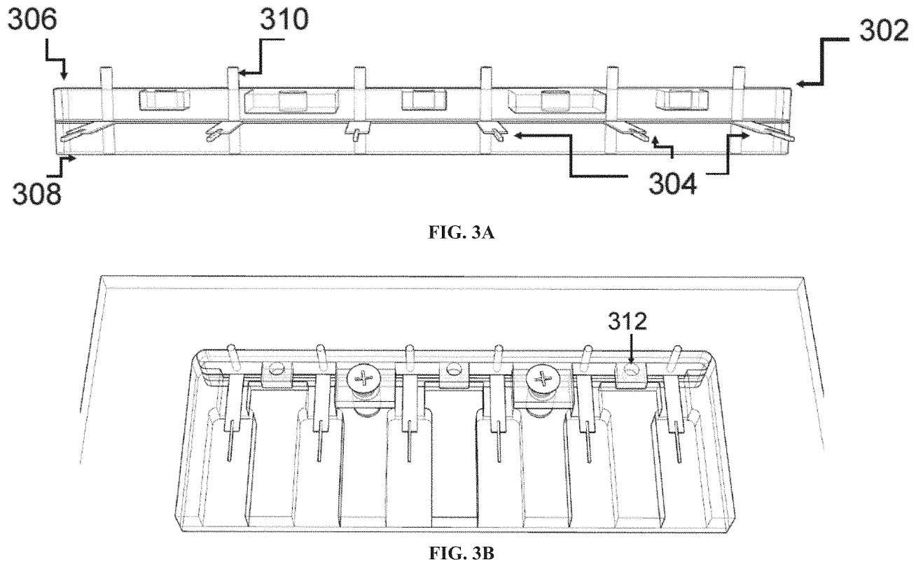

Referring now to FIG. 3A and 3B, an alignment component 302 for insertion of a set of sensors 304 in the engineered tissue strip module 102 is illustrated in accordance with embodiments of the present application. To properly align the set of sensors 304 within the engineered tissue strip module 102, the alignment component 302 may include a top frame 306 and a bottom frame 308 to secure multiple sensors within the same plane. In some embodiments, the set of sensors 304 may include two, three, four, five, six, seven, eight, nine, or ten sensors, or any integer greater than ten. For illustrative purpose, six sensors within the same plane is illustrated in FIG. 3A. In some embodiments, the set of sensors 304 and top/bottom frames may have corresponding holes or other registration mechanisms or markers for alignment purposes. To secure the frames and sensors 304 together, a dowel pin 310 may be press-fitted into each of the holes. Alternatively, the top frame 306 and the bottom frame 308 may be fastened by a screw or other tightening mechanism. Once assembled, the set of sensors 304 are secured in parallel in between the top frame 306 and bottom frame 308. Once the set of sensors 304 are secured, the alignment component 302 may be inserted into the engineered tissue strip module 102 in a cartridge-like mechanism and secured in place with another pair of alignment holes. It should be noted herein that the number of alignment holes should not be limited to two as more alignment holes may be included to further reduce any rotational offsets. In some embodiments, the top frame 306 of the alignment component 302 may have additional holes 312 between every two neighboring sensors that act as inlet or connectors for tubes of the perfusion system (not shown). The additional holes 312 may directly feed into a channel that connects or feeds into all of the multiple sensor lanes/channels in the engineered tissue strip module 102. The alignment component 302 may be made by laser cutting. The alignment component 302 may be fabricated from any biocompatible plastics, e.g. acrylic. The alignment component 302 may be made as a single-use or reusable item.

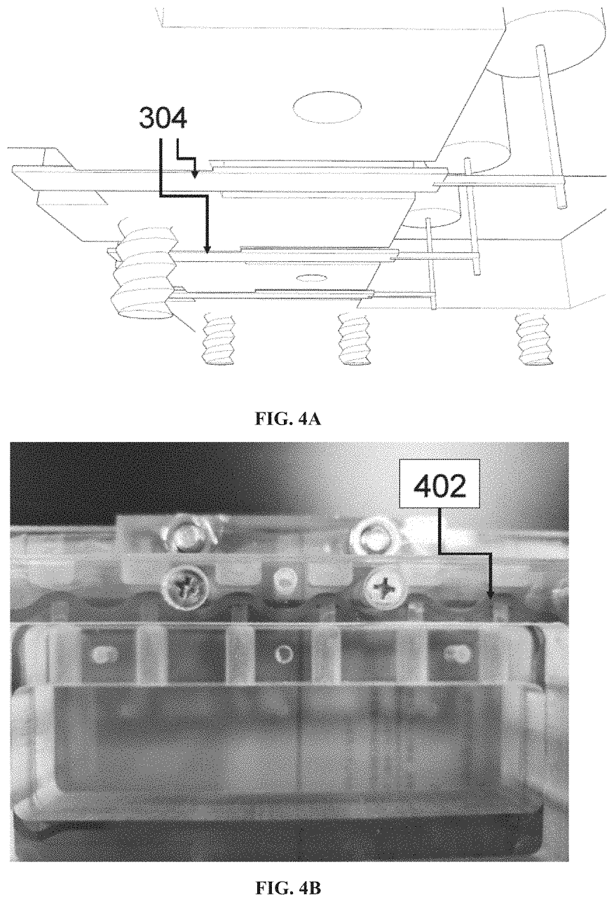

In some embodiments, as indicated in FIG. 4A and 4B, the sensors may be directly embedded onto the wall of the engineered tissue strip module 102, when there are regions of the sensors 304 which need to be isolated from liquid, especially when the sensors 304 are using an active sensing approach. A gasket 402 may be used to aid in creating a leakproof seal between the layers 210.

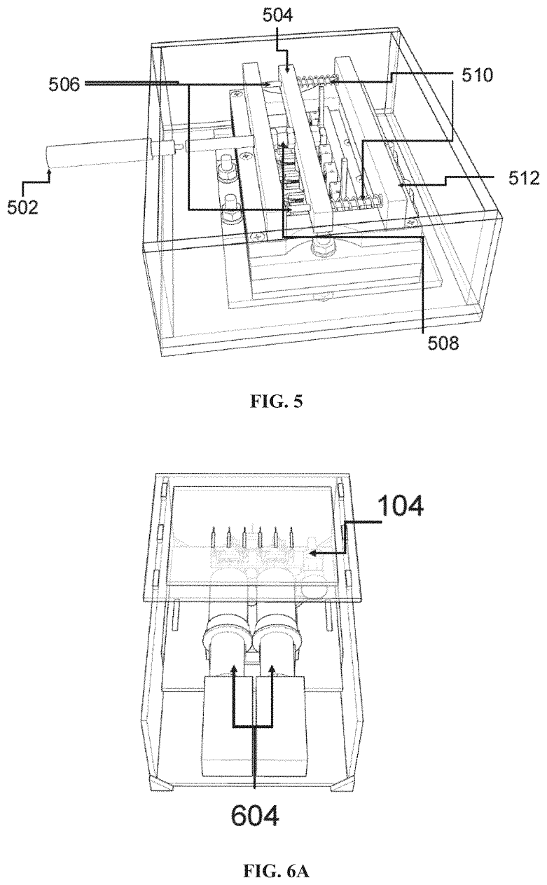

Referring now to FIG. 5, a 3D model of a tissue lengthening mechanism 108 is illustrated in accordance with the present application. The tissue lengthening mechanism 108 may be a linear actuator 502, in accordance with embodiments of the present application. The linear actuator 502 may be configured to displace forward and backward to allow lengthening and shortening of the engineered tissue strips. In order to assist the displacement of the movable block, a rail system 506 including one or more rods may be used. In some embodiments, the rail system 506 may include rails, tracks, bearings, and/or dovetails. For illustrative purpose, two rods are illustrated in FIG. 5. The rods restrict the movement of the movable block 504 to one axis. It should be appreciated that the rods can be of any shape including but not limited to square, triangular and circular. In some embodiments, compression springs 510 may be added between the movable block 504 and the rear support wall 512 so that the springs compress in response to the linear actuator extension, eliminating backlash and allowing the movable block 504 to return to position in response to the linear actuator retraction. It should be noted that the movable block 504 is secured to the rigid body 206, and the rigid body 206 is secured to one end of the engineered tissue strip as discussed above. Thus, the engineered tissue strips are stretched or shortened by the movement of the movable block 504. The movable block 504 may have a set screw 508 held in place using nuts. The set screw 508 is in line with the linear actuator 502 that is attached to the bioreactor wall and moves the movable block 504 in response to the extension/contraction of the linear actuator rod/shaft.

In some embodiments, the linear actuator 502 may be controlled by a custom software or a set of programmed instructions stored within the computer. The position of the linear actuator rod may be measured by the software and is used to calculate tissue length and percentage of tissue stretch. The linear actuator 502 can be configured to travel any distance desired. For example, the linear actuator 502 can travel a total distance of 2.54 cm (1 inch). In other embodiments, alternative mechanical devices, including those with motors and those without, can be utilized in place of linear actuator 502 as long as the alternative mechanical devices can move the moveable block 504 along an axis. In addition, the ratio of number of mechanical devices to number of engineered tissue strips may be altered. In one setup, each strip may have its own dedicated linear actuation 502 allowing complete custom stretch lengths for each tissue within the same engineered tissue strip module 102.

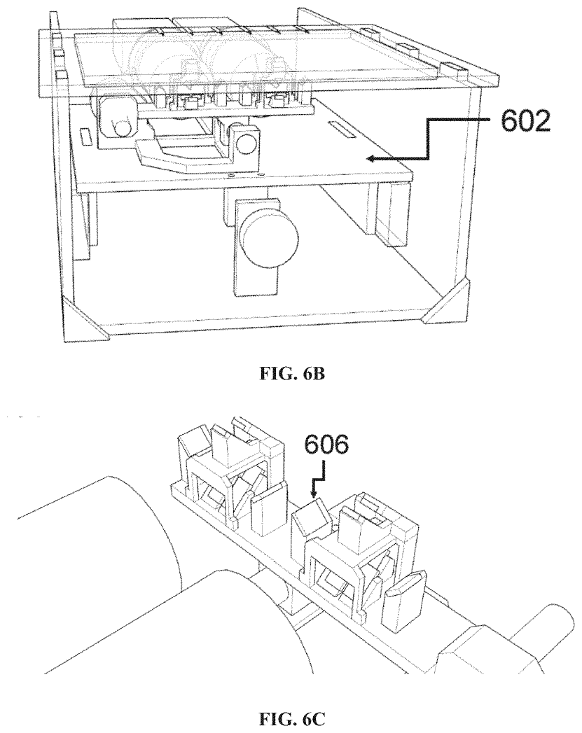

Referring now to FIG. 6A-6C, the mirror array system 104 for simultaneous acquisition of images/video of multiple engineered tissue strips is illustrated, in accordance with embodiments of the present application. The mirror array system 104 may be any arrangement of a mirror or mirrors that provides for the monitoring of a biological material, tissues, organoids, CTS or organs placed in each channel of the engineered tissue strip module 102. The mirror array system 104 enables use of fewer detection devices than the number of channels to improve the efficiency and lower the cost of monitoring. The mirror array system 104 may be a series of mirrors to enable imaging of more than one engineered tissue strips, placed in different channels of the engineered tissue strip module 102, using a single high-speed camera. The mirror array system 104 may enable an imaging platform 602 to optically measure twitch forces of multiple tissue strips simultaneously. Alternative mirror systems with arrangement of a mirror or mirrors that provides for the monitoring of multiple biological material (one or more cells, tissues, organoids or organs) are provided in WO2019106438A1, which is hereby incorporated by reference.

The imaging platform 602 may be built from any solid materials. In some embodiments, the imaging platform 602 may be built from a combination of laser cut plastic and 3D-printed parts. To perform inverted microscopy, the pair of cameras 112 with magnifying lenses may be oriented horizontally towards the front surface mirrors 606 that ultimately direct upwards toward the tissue strips. The mirrors 606 may be arranged to capture video/images of multiple engineered tissue strips simultaneously with a greater than 1:1 tissue: camera ratio (e.g. 3 tissues : 1 camera). The mirrors 606 may be designed to transmit separate views of the engineered tissue strips located relatively far apart and combine the views together into a single image captured by the pair of cameras 112. For example, each separate view may measure 3.54×10.62 mm (W×H). The entire field of view of the pair of cameras 112 may be a square image, e.g. a 10.62×10.62 mm square image. When using a camera with a 1024×1024 pixel readout, the imaging platform 602 may have a 10.4 microns per pixel resolution. In some embodiments, the mirrors 606 may be designed to have equal focal distance (for example 117 mm between sensor to the front of lens) for all tissues to ensure all engineered tissue strips are in the same focal plane. The platform may include multiple sliding elements that adjust the distance between the camera and the engineered tissue strips, effectively working together as a focusing mechanism to ensure that the engineered tissue strips are in focus with the pair of cameras 112. In some embodiments, the pair of cameras 112 are connected to a computer and controlled using the custom software. The program records images/video using the optical imaging platform, which are later processed into displacement and force measurements.

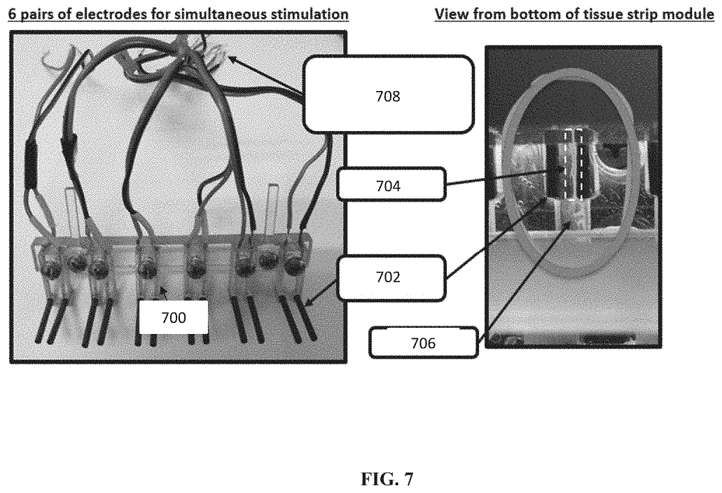

Referring now to FIG. 7, an electrode component 700 is illustrated in accordance with embodiments of the present application. In one embodiment, the electrode component 700 may be mounted close to the rigid body 206. The electrode component 700 may be composed of a conductive material or an array of electrodes 702 positioned on each side of each engineered tissue strip 704 to apply voltage for electrically pacing the engineered tissue strip 704 anchored to the sensor 706. Further, the electrode component 700 may act as a pacing system including the array of electrodes 702. The material of the electrodes, in the array of electrodes 702, may be selected from, but are not limited to, carbon, platinum, gold, or other conductive elements. The array of electrodes 702 may be positioned using an insert that is designed to fit the engineered tissue strip module 102. The array of electrodes 702 may be wired individually using a conductive element 708 such as platinum wires, which are wired to a programmable pulse generator (e.g. Master-9, AMPI).

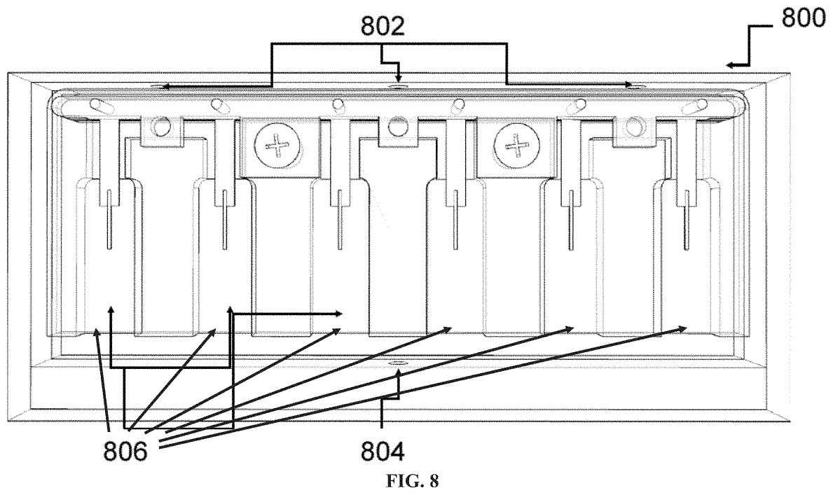

Referring now to FIG. 8, a top view of a perfusion system 800 is illustrated, in accordance with embodiments of the present application. During perfusion, a common inlet port 802 and a common outlet port 804 may be each connected to a tubing. In some embodiments, the common inlet port 802 may be connected to a Tygon 2-stop tubing with 0.89 mm ID, and the common outlet port 804 may be connected to a Tygon 2-stop tubing with 2.79 mm ID. In some embodiments, a pump (not shown) may be used to drive fluid flow into and suction out of the engineered tissue strip module 102. In some embodiments, the pump may be a multi-channel peristaltic pump. In some embodiments, drug-dosed media may be pumped into the engineered tissue strip module 102, preferably at flow rates ranging from 0.001 to 10 mL/min per channel, wherein the liquid height in each channel 806 is set by adjusting the position of the outlet tubing, wherein the outlet flow is recycled back to the inlet media reservoir or disposed of, depending on the experiment being run.

In some embodiments, a Masterflex 8-channel peristaltic pump (not shown) may be used to drive fluid flow into and suction out of the engineered tissue strip module 102. Each inlet port may be designed to feed a set of two channels. For each drug dose, a washout procedure with media containing no drug may be run after dosing. At the time of dosing, the pump may be paused, and inlet lines may be switched to a new media reservoir with drug premixed to the correct concentration prior to resuming flow.

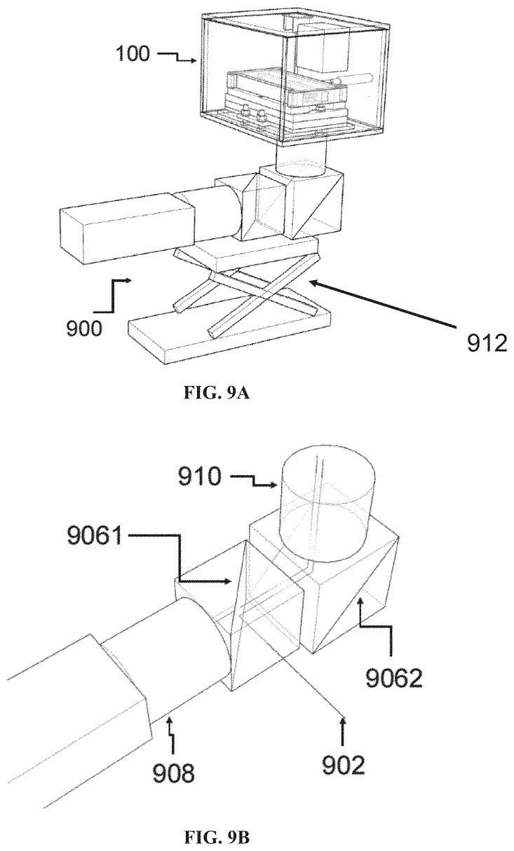

Referring now to FIG. 9, the multi-sampling system 100 is illustrated in accordance with embodiments of the present application. As shown, the multi-sampling system 100 may be enabled with an inverted optical mapping system 900. The inverted optical mapping system 900 may include an excitation light source 902, filter cubes, mirrors 9061, 9062, camera 908, lens 910, and adjustable laboratory jack 912. The inverted optical mapping system 900 may be configured to measure tissue properties including conduction velocity of the engineered tissue strips. The engineered tissue strips exhibit fluorescence or bioluminescence such as through the use of fluorescent or bioluminescent dyes or the expression of transgenes encoding fluorescent or bioluminescent proteins. In one embodiment, the tissue properties may be electrophysiological characteristics. The tissues may be injected with fluorescent dyes, such as voltage-sensitive dyes or calcium-sensitive dyes, which change in fluorescent intensity under different cellular conditions. Further, the excitation light source 902 may be any excitation light source known in the art, including but not limited to ultraviolet, blue, green, yellow, or red excitation light sources. Further, the filter boxes may be outfitted with a longpass dichroic mirror 9061 greater than 500 nm, and a wide-range mirror 9062.

In order to map conduction, tissues may be stimulated. Point stimulation may be achieved using a stimulation probe as a (i) substitute for or (ii) supplement to the rigid body 206. In some embodiments, the stimulation probe is a Microbes platinum/iridium concentric stimulation probe. When stimulation probe is used as substitute for the rigid body 206, the tissue is grown around the tip of the stimulation probe. When stimulation probe is used as supplement to the rigid body 206, the stimulation probe is carefully manipulated into a position that contacts or just penetrates the surface of the tissue. An alternative approach for stimulation may include isolated field stimulation where the anchoring needle is used as the positive terminal and a separate electrode negative terminal electrode is positioned in the media near the needle-anchored end of the tissue.

In some embodiments, each sensor may be marked with a marker. The marker may be a dot (such as SmoothOn Ignite Silicone dyes) that is detectable when excited by the same fluorescent light source used to measure electrophysiological characteristics and is therefore, also trackable during conduction velocity measurements. This marker may be tracked simultaneously to capture sensor movement. These displacements may be used to calculate the force applied by the tissue.

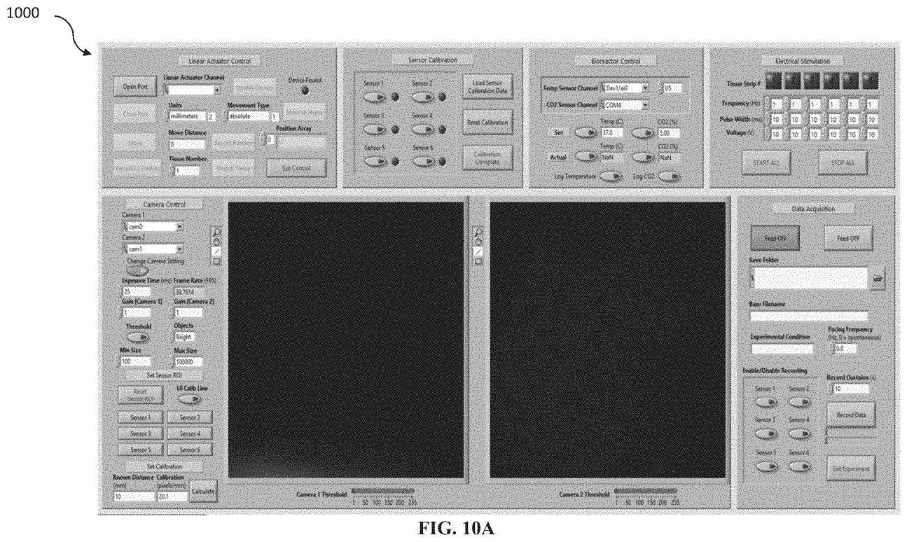

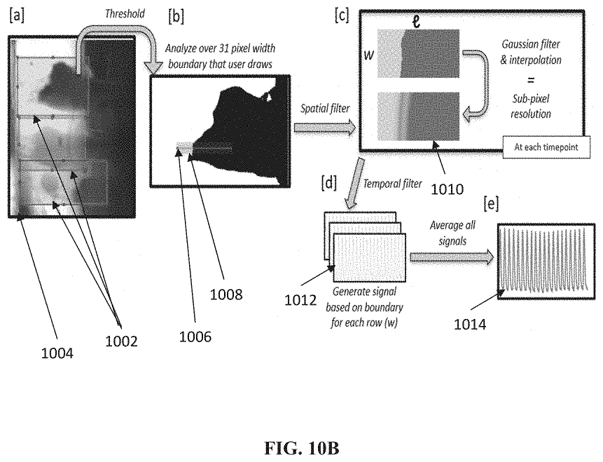

Referring now to FIG. 10A, a user interface 1000 of a Custom software for the bioreactor 106 is illustrated in accordance with embodiments of the present application. The custom software may be configured for capturing information from different sensors installed in the bioreactor 106 and control every component requiring electronic control within the bioreactor 106. Such components include linear actuator, heating unit, thermocouple (for measurement of target temperature, actual temperature, and the like), CO₂ sensor, solenoid valve (target CO₂ level, opening and closing of valve to flow in CO₂), pulse generator (voltage, frequency, pulse width), cameras (exposure time, gain), and the like. In addition, the software may be configured to perform several functions, including the creation of markers on the images acquired from the camera to allow user to set the linear actuator to the tissues at an unstretched length, calculation of tissue length and percentage stretch based on the linear actuator position, and saving and converting the images acquired from the camera into a video file using a semi-automated file naming scheme. Furthermore, the custom software may be configured to perform an image-processing based kymograph technique on the video captured by the custom software. The kymograph technique is further illustrated in FIG. 10B.

Referring now to FIG. 10B, the kymograph-based image processing technique is illustrated in accordance with embodiments of the present application. The imaging platform 602 allows imaging of multiple engineered tissue strips using a single camera. However, the number of pixels in a video frame corresponding to each engineered tissue strip are significantly reduced due to capturing of multiple engineered tissue strips in a single frame, since the mirror system works by splitting the field of view of the camera into smaller regions which are used to image each engineered tissue strip.

Due to this reason, getting all the engineered tissue strips in the field of view becomes a challenge. The sensor 204 helps resolve this issue, as the sensor increases the displacement signal of the tissue and thus allows a larger field of view (by lowering the magnification) without sacrificing resolution.

Furthermore, the engineered tissue strips may be in different focal planes, which can cause signal variability when tracking movement of the CTS. The image-processing based kymograph technique effectively solves this issue. The kymograph technique enables custom boundary tracking to derive signal of generated force with respect to time for each engineered tissue strip.

The kymograph technique involves steps of (a) defining a region-of-interest (ROI) for engineered tissue strip along with reference marks/line in each frame of a video captured by detection device, (b) applying a binary thresholding on each ROI and selecting a primary axis of the contractile motion of the engineered tissue strip, (c) applying a Gaussian filter in a spatial manner followed by interpolation, to the ROI at each timepoint of the video frame to give sub-pixel resolution, (d) generating a signal over time based on the sub-pixel resolution captured from each video frame, wherein the signal represents engineered tissue strip position over time, and (e) generating a representative signal by averaging all the signals generated from each target region in the video frame. Furthermore, a sensor is employed that incorporates a stretchable region that significantly increases the displacement of the sensor head for a given force. Each of these steps are further elaborated below.

In the first step, a region-of-interest (ROI) corresponding to each CTS from the set of CTSs or a subset of CTSs is defined in a frame of the video. The ROI 1002 is selected along reference marks/line 1004, wherein the reference marks/line 1004 corresponds to stationary objects in the bioreactor. In some embodiments, the custom software may allow the user to draw the ROI 1002 for each CTS visible in the field-of-view, and one or more reference marks/line 1004 of stationary objects in the bioreactor. As referenced, in FIG. 10B, a single best-fit line is drawn as the reference marks/line 1004 connecting the edges of the three wells that are reflected into a single image acquired by the camera. The absolute position of the reference mark 1004 should be known in order to accurately calculate the length of the engineered tissue strip. Furthermore, for each engineered tissue strip and the respective ROI 1002, binary thresholding is applied depending on the light intensity (converting the selected pixel values to either values of 0 or 1). For the purpose of binary thresholding, an ideal threshold is selected to distinctively isolate the boundary of the marker (on the sensor) or a distinct feature on the end of the engineered tissue strip. This thresholding step can be automated if the marker size is well established and tissue encapsulation or other obscuring of the marker area is prevented.

In the second step, a primary axis 1006 of CTS contraction and a target region 1008 along the primary axis 1006 of CTS contraction in the binary pixel representation of each ROI 1002 is defined. For this purpose, the user may draw a line that represents the primary axis 1006 of engineered tissue strip contraction. This step addresses any misalignment of either the acquisition setup or any off-axis motion caused by the engineered tissue strip. For the purpose of identifying the target region 1008, neighboring pixels of the primary axis 1006 are considered, e.g. a 31-pixel width boundary (extending 15 pixels to the left of the line and 15 to the right of the primary axis) as represented in FIG. 10B. This boundary width can be adjusted accordingly by the user with the smallest value for boundary width being 1. It should be understood that the target region 1008 may either correspond to boundary of the marker (on the sensor) or a distinct feature on the end of the CTS. In FIG. 10B, distinct feature at the end of the engineered tissue strip is considered as an example, however, marker on the sensor may also be considered for generating the binary pixel representation as long as the shape and color of the marker is known.

In the third step, gaussian filter and interpolation on to the target region 1008 is applied, in a spatial manner, on each video frame of the video to determine a sub-pixel resolution 1010 corresponding to the target region in each video frame. For example, if the video acquisition is regarded as a three-dimensional matrix of (width×length×time), a two-dimensional Gaussian filter is first applied to each time slice or point (width×length). Other two-dimensional image filters can be applied to smooth the boundary, especially if the pixel resolution is not high. Once the Gaussian filter is applied, each time slice is interpolated such that in between two pixels a number of (e.g. a hundred) new points are established. This interpolation step provides the ability to get sub-pixel, nanometer-scale resolution in an artificial manner when the camera's pixel resolution cannot.

In the fourth step, a signal 1012 over time is generated based on the sub-pixel resolution 1010 captured from each video frame, wherein the signal represents engineered tissue strip position over time. For this purpose, temporal filters may be applied to the three-dimensional matrix of the acquisition. This is done first by applying another binary threshold at each time slice of the now-Gaussian smoothed and interpolated matrix. Next, each row of a given time slice (or width, assuming that the primary axis of the contractile movement happens along the axis of the width) is examined by identifying the boundary of the tissue or marker by detecting the first change in pixel values along the length. This is repeated for all time slices to create multiple signals that represent spatial position over time, similar to a kymograph. For instance, in the default 31-pixel width boundary previously mentioned, 31 kymograph-like signals are generated. Each of these signals is then filtered temporally by using a locally weighted scatterplot smoothing filter. Other temporal filters, including but not limited to median, Savitzky-Golay, or moving-average filter, may also suffice.

In the fifth and final step, a representative signal 1014 is generated by averaging all the signals generated from each target region 1008 in the video frame. For this purpose, all of the filters are averaged to create a representative signal of the tissue position during the acquisition. By combining this signal with the reference line drawn in step one, the displacement of the end of the engineered tissue strip relative to a stationary reference is then derived. Using an established force-displacement relationship curve of the sensor (measured empirically or modeled), the generated force with respect to time can be output. This can be repeated for all engineered tissue strips within the field-of-view. This technique provides a way to acquire contractions of multiple engineered tissue strips in a simultaneous manner without limiting throughput. With the help of this technique a range of force detection was approximately 1 to 4000 μN. The limits can be altered significantly by changing the sensor properties.

### EXAMPLES

The following examples are provided to illustrate certain particular features and embodiments and should not be construed as limiting.

### Example 1: Preparation of Cell Solution and Seeding

A preparation of cell solution and seeding was prepared. For this purpose, pluripotent stem cell-derived cardiomyocytes were dissociated on day 15 using 0.025% Trypsin/EDTA and allowed to reaggregate in suspension in RPMI+B27 supplement (with ascorbic acid and ROCK inhibitors) for 72 hours prior to the day of seeding. Each tissue formed around a sensor utilized 1×10⁶ to 1.3×10⁶ hPSC-CMs. Human foreskin fibroblasts (HFF) were harvested from the culture plate using 0.05% TE. Each tissue formed around a sensor required 0.1×10⁶ to 0.13×10⁶ HFFs (10% of hPSC-CM number).

Further, the cellular mixture was formed by combining the following components: 40% of 5 mg/ml collagen, 1.5% 1M NaOH, 9% 10X MEM, 12.5% 0.2M HEPES, 10% DMEM with newborn calf serum (NCS, 10%) and 6-10% Matrigel, then replenished by ultrapure water to 100%. This collagen mixture was added to the cell mixture (hPSC-CM+HFF) and the total volume was brought up to 180 μL per tissue using NCS media. This solution was used for seeding by pipetting 180 μL of cell solution into each sensor section (avoiding bubble formation) that had been defined by the PDMS inserts described above. The engineered tissue strip module 102 was then placed in the incubator for 1 hour prior to topping up with media by adding 30 mL NCS to the engineered tissue strip module 102. Tissues were allowed to compact around sensors over the next 2-3 days prior to removing PDMS inserts. Tissues attached to sensors were ready for testing 7 days after seeding as represented in FIG. 7.

### Example 2: Sensor Preparation for Tissue Formation

A sensor preparation for tissue formation was assembled and placed in the bioreactor. Prior to seeding, sensors were washed in a soapy water bath with agitation to remove residues that may have accumulated from fabrication. All sensors were then soaked in 70% ethanol. Sensors were removed from ethanol and positioned on an acrylic slab such that the sensor heads just extended over the edge of the slab. A layer of medical adhesive backing was applied to cover the sensor bodies and secured with masking tape such that the sensor heads were exposed on both sides. The sensors were UV ozone treated for 30 minutes. The sensors are then fixed by clamping the sensors in between two solid layers with the tissue attachment area and placed into a well or trough. PDMS inserts were used to limit the volume around the sensors such that the tissues were able to encapsulate the sensor heads. Before seeding, 180 μL of 2% BSA in PBS solution was added to each sensor well/channel, avoiding bubble formation. The engineered tissue strip module 102 was incubated at 37° C., 5% CO₂ for 1 hour then the BSA solution was aspirated. The bioreactor 106 was allowed to air dry prior to seeding.

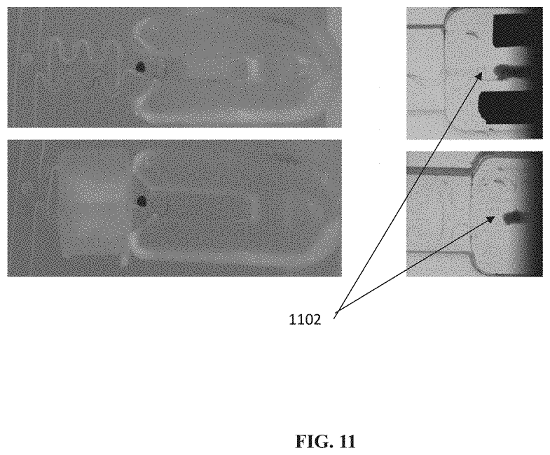

FIG. 11 illustrates the sensors used within the bioreactor module. PDMS inserts were used during the initial tissue formation (left). The tissue anchored around the opening of the sensor end 1102.

### Example 3: Length-Tension Relationship

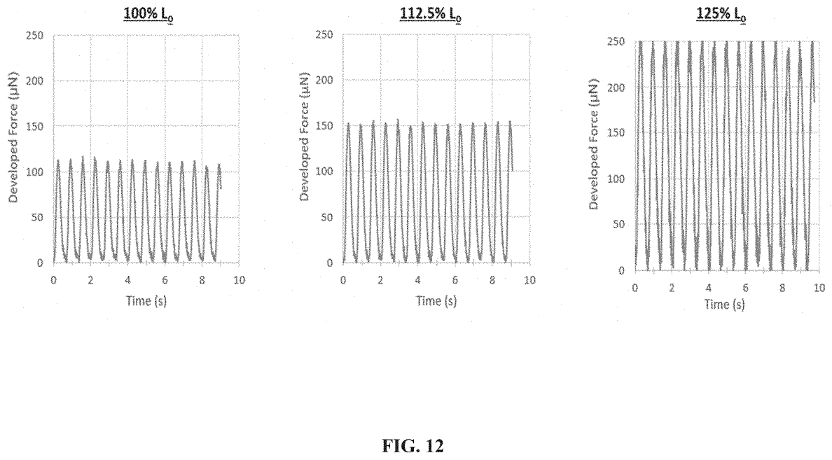

Referring now to FIG. 12, graphs representing a positive length-tension relationship are illustrated in accordance with an exemplary embodiment of the present application. For this purpose, CTSs as the engineered tissue strips were placed within the engineered tissue strip module 102 to demonstrate the capability of the multi-sampling system 100 in capturing the length-tension relationship of the CTSs. At day 7 post-seeding, the CTSs were stretched to a length that was 125% of the initial length. As represented in the graphs of FIG. 12, as the CTSs were extended the associated developed force increased by over two-fold. This trend is a result due to an increase in the number of actin-myosin crossbridges formed when the tissue is stretched. This results in an increase in the force generated when the CTS or muscle contracts.

### Example 4: Sensitivity to Cell-Line Variability and Disease Phenotype

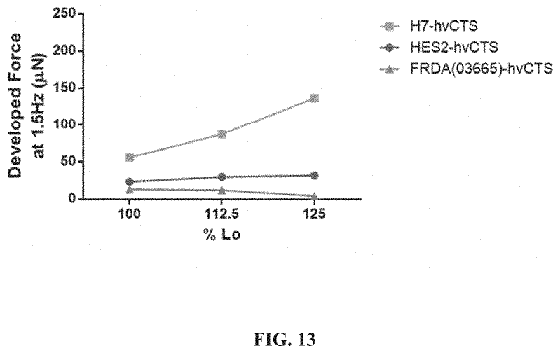

Sensitivity of the multi-sampling system 100 to cell-line variability and disease phenotype was examined. For this purpose, CTSs from three different cell lines, including one representing a diseased phenotype (Friederich's Ataxia), were installed in the engineered tissue strip module 102 to validate the capability of the multi-sampling system 100 to utilize both human embryonic stem cell and human induced pluripotent stem cell lines and subsequently measure various force ranges. As represented by the graph in FIG. 13, strips composed of human stem cell-derived cardiomyocytes from the H7 line had the highest developed force along with the biggest increase in force when lengthened to 125% of the strips' initial lengths. Further, the CTSs from the HES2 line also demonstrated positive length-tension relationship on a smaller force range, whereas the tissue strips from the Friederich's Ataxia line, FRDA (03665), demonstrated the lowest contractility and decreased in developed force when stretched to 125% of initial length.

### Example 5: Screening of Drug Compounds

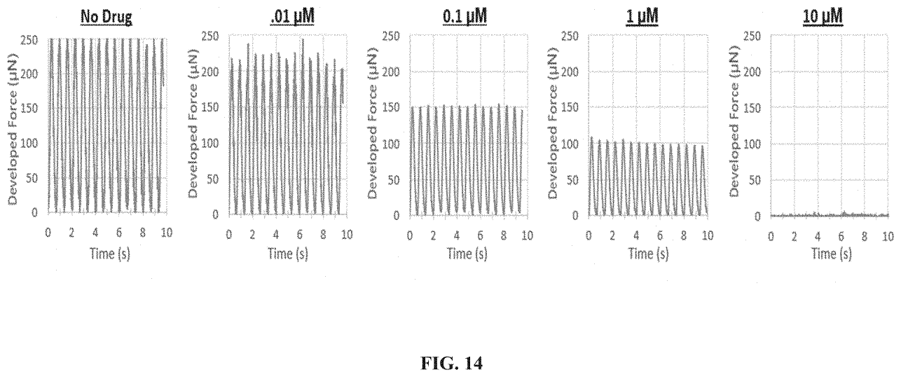

One of the functionalities of the multi-sampling system 100 is screening of drug compounds. To demonstrate this, the pharmacological response of CTSs to the exposure of nifedipine (a known negative inotrope) was captured as represented in FIG. 14. At the highest concentration of 10 μM of nifedipine, CTSs stopped exhibiting any contractions or developed force. The effects of the drug can be seen even at the lowest concentration of 0.01 μM and demonstrate a concentration-dependent response. Such drug testing may be performed with varying degrees of stretch of the CTSs, as enabled by the tissue lengthening mechanism. The stretching allows a simulation of the physiological state of cardiac muscle as the heart fills with blood, and an increase in contractile force as predicted by the Frank-Starling law of the heart. Such an increase in force can aid detection of drug effects on CTS contractility.

Although implementations of the multi-sample system for CTS assays have been described in language specific to structural features, it is to be understood that the appended claims are not necessarily limited to the specific features. Rather, the specific features are disclosed as examples of implementations of the multi-sample system for engineered tissue strip assays.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A multi-sample system for engineered tissue strip assay comprising:
an engineered tissue strip module enabled with a set of channels, wherein each channel is configured to house one or more engineered tissue strips from a set of engineered tissue strips, wherein each engineered tissue strip is in contact with a sensor at one end and a tissue lengthening mechanism at the other end, wherein the tissue lengthening mechanism is configured to lengthen or contract at least one of the engineered tissue strips, wherein the sensor is displaced or deformed when the engineered tissue strip exerts force against the sensor during the lengthening or contraction of each of the engineered tissue strips; and
a detection system configured to capture change in the sensor in contact with the engineered tissue strip, during lengthening or contraction of at least one of the engineered tissue strips.

2. The multiple-sample system as claimed in claim 1, wherein the detection system comprises a mirror array system comprising an imaging platform with a camera and a set of mirrors, wherein the camera is configured to capture a video of at least one of the engineered tissue strips and the sensor in contact with the engineered tissue strip, through the set of mirrors, during the lengthening or contraction of at least one of the engineered tissue strips.

3. The multi-sample system as claimed in claim 2, wherein the video is processed using an image-processing based kymograph for determining characteristic properties of each of the engineered tissue strips from the set of engineered tissue strips or the subset of engineered tissue strips.

4. The multi-sample system as claimed in claim 2, further comprising a bioreactor for housing the engineered tissue strip module and the mirror array system, wherein the bioreactor provides environmental control and monitoring of one or more environmental conditions of interest comprising temperature, carbon dioxide (CO₂), relative humidity and oxygen (O₂) concentration in the bioreactor.

5. The multi-sample system as claimed claim 4, wherein the tissue lengthening mechanism is configured to control linear displacement.

6. The multi-sample system as claimed in claim 5, wherein the tissue lengthening mechanism comprises a linear actuator, a micrometer, a motor, a ratchet, a pinion, a camshaft, or a mechanical linkage integrated into the bioreactor.

7. The multi-sample system as claimed in claim 1, wherein the engineered tissue strip module enables formation, culturing, and controlling of at least one of the engineered tissue strips, and wherein each channel is enabled with an inlet port and an outlet port, wherein a perfusion system is configured for fluid circulation in each channel through the inlet port and the outlet port, and wherein each channel is recessed to allow for the fluid to flow around the engineered tissue strips wherein volume of the fluid is limited and contained in specific part of the set of channels with the sensor, wherein at least one of the set of channels are connected upstream and downstream to common areas for inlet and outlet of fluid, and wherein the common areas are connected to the perfusion system through a common inlet port and common outlet port.

8. The multi-sample system as claimed in claim 7, wherein the set of channels is isolated from each other with a dedicated inlet and outlet for each channel, and wherein the dedicated inlet and outlet of each of the channel connects to a same or different perfusion system.

9. The multi-sample system as claimed in claim 1, wherein one end of each of the engineered tissue strips is anchored to a rigid body that the engineered tissue strips cannot displace or deform, wherein the rigid body is selected from a shape comprising needle, rod, pin, post, or anchor, and wherein the rigid body is connected to a movable block of a rail system to enable lengthening of the engineered tissue strips.

10. The multi-sample system as claimed in claim 9, wherein the rigid body is enabled with an electrode component configured to electrically pace the set of engineered tissue strips or the subset of engineered tissue strips, wherein the electrode component is composed of a conductive material or an array of electrodes positioned on each side of the engineered tissue strips to apply voltage/current for electrically pacing the engineered tissue strips, and wherein the rigid body is enabled to aid in the anchoring of the engineered tissue strips.

11. The multi-sample system as claimed in claim 1, wherein the sensor is a geometrically defined material that displaces and/or deforms when the engineered tissue strip exerts force on the sensor, and wherein the displacement and/or deformation of the sensor is monitored by the detection system to measure the lengthening or contraction of the engineered tissue strip, wherein the sensor is a passive sensor or an active sensor.

12. The multi-sample system as claimed in claim 1, further comprising an alignment component to align each of the sensors in the channels of the engineered tissue strip module, wherein the alignment component comprises a top frame and a bottom frame configured to accommodate the sensors in between the top frame and bottom frame, wherein the top frame, bottom frame and sensors have corresponding holes or registration mechanisms or markers that are used for alignment of the sensor in between the top frame and the bottom frame within the same plane, wherein a dowel pin is press-fitted into each of the holes to align the sensors, wherein the top frame of the alignment component have additional holes between every two sensors, wherein the additional holes act as inlet for tubes of the perfusion system, and wherein the additional holes directly feed into the channels of the engineered tissue strip module.

13. The multi-sample system as claimed in claim 1, wherein the sensors are directly embedded into the wall of the engineered tissue strip module when at least one region of the sensors is to be isolated from a fluid reservoir.

14. The multi-sample system as claimed in claim 9, wherein the tissue lengthening mechanism is configured to displace the movable block forward and backward to allow lengthening and shortening of the engineered tissue strip, wherein the rail system comprises one or more rods restricting movement of the movable block to one axis.

15. The multi-sample system as claimed in claim 2, wherein the at least one camera of the imaging platform is configured to optically measure twitch forces of the set of engineered tissue strips simultaneously, wherein the at least one camera is enabled with microscopic lenses oriented horizontally towards the set of mirrors that direct toward the engineered tissue strips, wherein the set of mirrors are arranged to enable the camera to capture multiple engineered tissue strips simultaneously, wherein the mirror arrangement enables capturing separate views of the engineered tissue strips located apart and combine the views together into a single image captured by the camera, wherein the set of mirrors are positioned to have equal focal distance for all of the engineered tissue strips to ensure all engineered tissue strips are in the same focal plane, wherein the set of mirrors are mounted over sliding elements that adjust the distance between the camera and the engineered tissue strip, and wherein the sliding elements enable a focusing mechanism to ensure that the engineered tissue strips are in focus with the camera.

16. The multi-sample system as claimed in claim 7, wherein a multi-channel peristaltic pump is used to drive fluid flow into and suction out of the engineered tissue strip module, wherein fluid is pumped into the engineered tissue strip module at flow rates ranging from 0.001 to 10 mL/min per channel, wherein liquid height in each channel is set by adjusting the position of an outlet tubing, and wherein the outlet flow is recycled back to an inlet of the fluid reservoir, collected for analysis, redirected for collection or disposed of.

17. The multi-sample system as claimed in claim 2, wherein the imaging platform is enabled with an optical mapping system, wherein the optical mapping system comprises an excitation source, filter cubes, mirrors, camera, and lens, wherein the optical mapping system is configured to measure tissue properties including conduction velocity of the engineered tissue strips, wherein the engineered tissue strips exhibit fluorescence or bioluminescence.

18. The multi-sample system as claimed in claim 17, wherein each of the sensors is marked with a marker, wherein the marker is detectable when excited by fluorescent light source, wherein the marker is trackable during measurements, and wherein the marker is tracked simultaneously to capture sensor movement, wherein the sensor movement is used for computing applied force by the tissue.

19. The multi-sample system as claimed in claim 6, further comprising program executable instructions for controlling the linear actuator, a heating unit, a thermocouple, a CO₂ sensor solenoid valve, and cameras, the program executable instructions comprising instructions to:
create markers on the images acquired from the camera to allow a user to set the linear actuator to the engineered tissue strips at an unstretched length,
calculate tissue length and percentage stretch based on the linear actuator position, and
save and convert the images acquired from the camera into a video file using a semi-automated file naming scheme.

20. The multi-sample system as claimed in claim 3, wherein the image-processing based kymograph comprises the steps of:
defining a region-of-interest (ROI) corresponding to each of the engineered tissue strips from the set of engineered tissue strips or the subset of engineered tissue strips, in a frame of the video, wherein the ROI is selected along reference marks, wherein the reference marks corresponds to stationary objects in the bioreactor;
applying binary thresholding on each pixel in each of the ROIs to convert the ROI into a binary pixel representation;
defining a primary axis of the engineered tissue strip contraction and a target region along the primary axis of the engineered tissue strip contraction in the binary pixel representation of each of the ROIs;
applying Gaussian filter and interpolation on to the target region, in a spatial manner, of each video frame of the video, to achieve a sub-pixel resolution corresponding to the target region in each video frame;
generating a signal over time based on the sub-pixel resolution captured from each of the video frames, wherein the signal represents the engineered tissue strip position over time; and
generating a representative signal by averaging all the signals generated from each target region in the video frame.
